(19) United States
(12) Patent Application Publication
(10) Pub. No.: US 2026/0047946 A1
(43) Pub. Date: Feb. 19, 2026

(54) STENT WITH REINFORCED FLARED REGION

(71) Applicant: BOSTON SCIENTIFIC SCIMED, INC., Maple Grove, MN (US)

(72) Inventors: FIONN STAPLETON, Galway (IE); JOHN THOMAS O'DRISCOLL, Galway (IE); MICHAEL WALSH, Tuam (IE); SANDRA BURKE, Galway (IE); ENDA HANNON, Galway (IE); JONATHAN ROOT, Townsend, MA (US)

(73) Assignee: BOSTON SCIENTIFIC SCIMED, INC., Maple Grove, MN (US)

(21) Appl. No.: 19/301,254

(22) Filed: Aug. 15, 2025

Related U.S. Application Data
(60) Provisional application No. 63/683,885, filed on Aug. 16, 2024.

(51) Int. Cl. A61F 2/90

(52) U.S. Cl. CPC A61F 2/90; A61F 2230/0006; A61F 2230/0069; A61F 2250/0026; A61F 2250/0037; A61F 2250/0039

(57) ABSTRACT

A stent, such as transluminal stent, is disclosed. The stent includes a tubular member with a proximal end, a distal end, and a central portion therebetween. The elongate tubular member comprises a scaffolding forming a plurality of cells and defining a lumen of the elongate tubular member. A proximal portion of the tubular member forms a retention member, wherein the diameter of the retention member is greater than the diameter of the central portion, and wherein a proximal end of the retention member includes an area of increased scaffolding density.

### CROSS-REFERENCE TO RELATED APPLICATIONS

The application claims the benefit of U.S. Provisional Patent Application Ser. No. 63/683,885, filed on Aug. 16, 2024, the disclosure of which is incorporated herein by reference.

### TECHNICAL FIELD

The disclosure pertains to methods and apparatuses for various ailments. More particularly, the disclosure is related to different configurations, methods of manufacture, and use of a stent.

### BACKGROUND

Implantable stents are devices that are placed in a body structure or lumen, such as a blood vessel, esophagus, trachea, biliary tract, colon, intestine, stomach or body cavity, to provide support and to maintain patency of the structure or to maintain patency between two structures that have been connected via a stent for an alternate drainage path (e.g. hepaticogastrostomy). These devices are manufactured by any one of a variety of different manufacturing methods and may be used according to any one of a variety of methods for a variety of applications. Of the known medical stents, delivery systems, and methods, each has certain advantages and disadvantages. For example, some stents may migrate after implantation. There is an ongoing need to provide alternative stents, as well as alternative methods for manufacturing and using the stents.

### SUMMARY

This disclosure provides design, material, manufacturing method, and use alternatives for medical devices. An example medical device includes a transluminal implant or stent.

A first example is a stent including an elongate tubular member having a first end, a second end, and a lumen extending from the first end to the second end. The tubular member includes a scaffolding formed of a plurality of interwoven wires defining a plurality of interstices, a first end region extending to the first end, a second end region extending to the second end, and a body region extending between the first end region and the second end region. The second end region forms a retention member. A diameter of the retention member is greater than a diameter of the body region. A circumferential rim of the retention member includes an area of increased scaffolding density relative to a remainder of the first end region.

Alternatively or additionally to any of the examples herein, in another example, the area of increased scaffolding density forms an annular ring of the interwoven wires disposed around an outer circumference of the second end of the tubular member.

Alternatively or additionally to any of the examples herein, in another example, the annular ring of interwoven wires includes a plurality of wire loops extending around the circumferential rim.

Alternatively or additionally to any of the examples herein, in another example, each of the plurality of wire loops includes an apex and a base opposite the apex, wherein the base is a cross-over point of a pair of the plurality of interwoven wires.

Alternatively or additionally to any of the examples herein, in another example, the apex of each of the plurality of wire loops is juxtaposed with the base of the associated wire loop.

Alternatively or additionally to any of the examples herein, in another example, each of the plurality of wire loops has an as-braided configuration in which the apex is spaced apart from the base of the associate wire loop by a first distance, and wherein each of the plurality of wire loops has a heat-set configuration in which the apex is juxtaposed with the base of the associated wire loop a second distance, wherein the first distance is greater than the second distance.

Alternatively or additionally to any of the examples herein, in another example, the apexes of the plurality of wire loops are arranged around the circumferential rim.

Alternatively or additionally to any of the examples herein, in another example, the circumferential rim has a first diameter in the as-braided configuration and a second diameter in the heat-set configuration, wherein the second diameter is greater than the first diameter.

Alternatively or additionally to any of the examples herein, in another example, the plurality of wire loops defines an end-most circumferential row of interstices, wherein the interstices in the end-most circumferential row of interstices have a smaller cross-sectional area than interstices along the body region.

Alternatively or additionally to any of the examples herein, in another example, the area of increased scaffolding density includes an additional wire circumferentially woven into the scaffolding around the circumferential rim.

Alternatively or additionally to any of the examples herein, in another example, the retention member is substantially flange shaped.

Alternatively or additionally to any of the examples herein, in another example, the first end region of the tubular member forms a retention member, wherein a diameter of the retention member of the first end region is greater than the diameter of the body region.

Alternatively or additionally to any of the examples herein, in another example, a circumferential rim of the retention member of the first end region includes an area of increased scaffolding density.

Another example is a stent including an elongate tubular member having a first end, a second end, and a lumen extending from the first end to the second end. The tubular member includes a scaffolding formed of a plurality of interwoven wires defining a plurality of interstices. The tubular member includes a first end region extending to the first end, a second end region extending to the second end, and a body region extending between the first end region and the second end region. The second end region is a flared region tapered radially outward to an outermost circumferential rim at the second end. A diameter of the circumferential rim is greater than a diameter of the body region. The plurality of interwoven wires forms a plurality of wire loops extending around the circumferential rim. Each of the plurality of wire loops includes an apex and a base opposite the apex, wherein the base is a cross-over point of a pair of the plurality of interwoven wires. The apex of each of the plurality of wire loops is juxtaposed with the base of the associated wire loop.

Alternatively or additionally to any of the examples herein, in another example, the plurality of wire loops defines an end-most circumferential row of interstices, wherein the interstices in the end-most circumferential row of interstices have a smaller cross-sectional area than interstices along the body region.

Alternatively or additionally to any of the examples herein, in another example, each of the plurality of wire loops has an as-braided configuration in which the apex is spaced apart from the base of the associate wire loop by a first distance, and wherein each of the plurality of wire loops has a heat-set configuration in which the apex is juxtaposed with the base of the associated wire loop a second distance, wherein the first distance is greater than the second distance.

Alternatively or additionally to any of the examples herein, in another example, the apexes of the plurality of wire loops are arranged around the circumferential rim.

Alternatively or additionally to any of the examples herein, in another example, the circumferential rim has a first diameter in the as-braided configuration and a second diameter in the heat-set configuration, wherein the second diameter is greater than the first diameter.

Another example is a method of forming a stent. The method includes: braiding a plurality of wires to form a tubular scaffolding comprising a plurality of loops arranged around a circumferential rim of the tubular scaffolding; placing a cap over the circumferential rim of the tubular scaffolding; compressing the plurality of loops arranged around the circumferential rim of the tubular scaffolding to form a condensed wire mass around the circumferential rim of the tubular scaffolding; heat setting the condensed wire mass; and removing the cap.

Alternatively or additionally to any of the examples herein, in another example, each of the plurality of wire loops includes an apex and a base opposite the apex, wherein the base is a cross-over point of a pair of the plurality of interwoven wires. The apex of each of the plurality of wire loops is juxtaposed with the base of the associated wire loop after the heat setting step.

Another example is a stent including a tubular member having a proximal end, a distal end, and a body region therebetween. The elongate tubular member includes a scaffolding forming a plurality of cells and defining a lumen of the elongate tubular member. A proximal portion of the tubular member forms a retention member, wherein the diameter of the retention member is greater than the diameter of the body region. A proximal end of the retention member includes an area of increased scaffolding density.

Alternatively or additionally to any of the examples herein, in another example, the scaffolding comprises a shape memory wire.

Alternatively or additionally to any of the examples herein, in another example, the scaffolding includes a plurality of substantially repeating bent wire segments forming the plurality of cells.

Alternatively or additionally to any of the examples herein, in another example, the area of increased scaffolding density forms a ring disposed on the outer circumference of the proximal end of the tubular member.

Alternatively or additionally to any of the examples herein, in another example, the area of increased scaffolding density includes additional shape memory wire woven into the scaffolding.

Alternatively or additionally to any of the examples herein, in another example, the repeating bent wire segments at the proximal end of the tubular member are compressed to form the area of increased scaffolding density.

Alternatively or additionally to any of the examples herein, in another example, the stent has a constrained configuration and an unconstrained configuration.

Alternatively or additionally to any of the examples herein, in another example, the stent is self-expanding.

Alternatively or additionally to any of the examples herein, in another example, the retention member is substantially flange shaped.

Alternatively or additionally to any of the examples herein, in another example, wherein the tubular member comprises a coating or covering extending along at least a partial axial length of the tubular member.

Another example is a stent including a tubular member having a proximal end, a distal end, and a body region therebetween. The elongate tubular member includes a scaffolding forming a plurality of cells and defining a lumen of the elongate tubular member. The scaffolding includes a plurality of substantially repeating bent wire segments forming the plurality of cells. A proximal portion of the tubular member forms a retention member, wherein the diameter of the retention member is greater than the diameter of the body region. A proximal end of the retention member includes a ring of increased scaffolding density disposed on the outer circumference of the proximal end of the tubular member.

Alternatively or additionally to any of the examples herein, in another example, the scaffolding comprises a shape memory wire.

Alternatively or additionally to any of the examples herein, in another example, the area of increased scaffolding density includes additional shape memory wire woven into the scaffolding.

Alternatively or additionally to any of the examples herein, in another example, the repeating bent wire segments at the proximal end of the tubular member are compressed to form the area of increased scaffolding density.

Alternatively or additionally to any of the examples herein, in another example, the stent has a constrained configuration and an unconstrained configuration.

Alternatively or additionally to any of the examples herein, in another example, the stent is self-expanding.

Another example is a method of forming a stent. The method includes: weaving at least one wire to form a scaffolding comprising a plurality of loops, wherein the scaffolding forms a tubular member; weaving the at least one wire around a shaping member to form a flared portion at a proximal end of the tubular member; removing the shaping member; loading at least a portion of the flared portion into a cap, wherein the cap compresses the portion of the flared portion loaded within the cap; heating the cap to heat set the compressed portion of the flared portion; and removing the cap.

Alternatively or additionally to any of the examples herein, in another example, the wire comprises a shape memory wire.

Alternatively or additionally to any of the examples herein, in another example, the stent has a constrained state and an unconstrained state.

Alternatively or additionally to any of the examples herein, in another example, the method includes coating at least a portion of an axial length of the tubular member.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates an example medical stent;

FIG. 2 illustrates an example medical stent;

FIG. 3 illustrates a perspective view of the stent of FIG. 2;

FIG. 4A shows an example medical stent being woven around a shaping member;

FIG. 4B shows the medical stent of FIG. 4A with the shaping member removed;

FIG. 4C shows the medical stent of FIG. 4B being compressed into a cap; and

FIG. 4D shows the medical stent of FIG. 4C after heat treating.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following description should be read with reference to the drawings, which are not necessarily to scale, wherein similar elements in different drawings are numbered the same. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

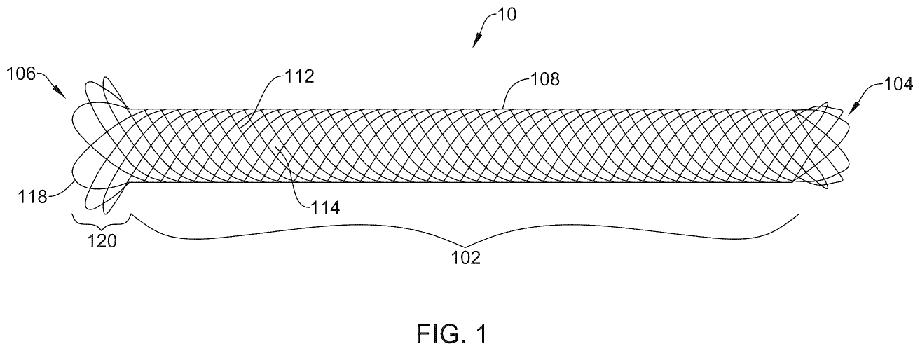

Patients with difficult anatomy, blockages or restrictions that cause failed endoscopic retrograde cholangiopancreatography (ERCP) are traditionally treated with hepaticogastrostomy (HGS), a biliary drainage conduit between the liver and the stomach. Stent migration out of the stomach is a severe risk associated with HGS stenting, which can result in leakage and tissue damage (e.g. biliary peritonitis). A method and device for preventing migration of a stent during HGS stenting procedures is needed. FIG. 1 provides a side view of an illustrative stent 10 for use in fluidly coupling two different body lumens, such as, but not limited to, a transluminal stent, in an expanded state. The stent 10 of FIG. 1 includes a first end 104, second end 106, and a central or body region 102 extending therebetween. The stent 10 may include a lumen extending from an opening near the first end 104 to an opening near the second end 106 to allow for the passage of bile, fluids, and the like therethrough. The stent 10 may further include a shoulder portion or flared region 120 having a diameter larger than the diameter of the central or body region 102. In some embodiments, the shoulder portion or flared region 120 may be provided at the second end 106 as shown in FIG. 1. Alternatively, the shoulder portion or flared region 120 may be provided at the first end 104, or on both the first and second ends 104, 106, if desired. There may be any number of shoulder portions or flared regions 120 disposed at different locations along the length of the stent. While the stent 10 described herein is generally tubular it is contemplated that the stent 10 may take any cross-sectional shape desired. The shoulder portion or flared region 120 is shown herein as having a generally tapered shape in which the tubular scaffold defining the stent 10 tapers radially outward toward the second end 106, but may take any cross-sectional shape desired.

Additionally, the shoulder region or flared region 120 may have a varying diameter and/or length depending on the anatomy of the lumen within which the stent 10 will be placed.

The stent 10 may be formed of a tubular member 108, which may be radially expandable from a first radially collapsed configuration (not explicitly shown), to a second radially expanded configuration, as shown in FIG. 1. The stent 10 may be structured to extend across two non-adherent structures/tissues and to apply a radially outward pressure to create an opening or passage between the two non-adherent structures/tissues, thereby forming an anastomosis between the two separate anatomical structures.

The tubular member 108 of the stent 10 may have a scaffold structure, fabricated from one or more, or a plurality of interwoven filaments 112 defining open cells or interstices 114 therebetween. The scaffold structure may extend from the first end 104 to the second end 106 of the stent 10 and define both the central or body region 102 and the shoulder portion or flared region 120. For example, the scaffold structure, and thus the filament(s) thereof 112, may extend continuously from the first end 104 to the second end 106 of the stent 10. In some embodiments, the stent 10 may be formed with one filament 112 interwoven with itself (e.g., knitted) to form the scaffold structure. In other embodiments, the stent 10 may be formed with several interwoven filaments 112 (e.g., braided, woven, interlocked, etc.) to form the scaffold structure. Thus, in such instances one or more of the filament(s) 112 forming the scaffold structure may extend continuously from the first end 104 to the second end 106 of the stent 10. In still another embodiment, the stent 10 may include a laser cut tubular member to form the scaffold structure. A laser cut tubular member may have an open and/or closed cell geometry including one or more interconnected struts formed as a monolithic structure from the tubular member. In such instances, the laser cut tubular member forming the scaffold structure may extend continuously from the first end 104 to the second end 106 of the stent 10.

In some instances, an inner and/or outer surface of the scaffold structure of the stent 10 may be entirely, substantially or partially, covered with a polymeric covering or layer. For example, a covering or coating may extend across the open cells 114 of the scaffold structure to prevent tissue ingrowth into the lumen of the stent. In some cases, the covering or coating may prevent food or particulate ingress into the lumen or fluid leakage from the lumen along the coated regions. While not explicitly shown, the covering or coating may include an outer layer disposed over an outer surface of the scaffold structure and/or an inner layer disposed over an inner (e.g., luminal) surface of the scaffold structure. In some embodiments, the stent 10 may include only an outer polymeric covering on an outer surface of the scaffold structure. In other embodiments, the stent 10 may include only an inner polymeric covering on an inner surface of the scaffold structure. In some instances, an inner layer and an outer layer may be formed as a single unitary structure to form the covering or coating. In other embodiments, an inner layer and an outer layer may be formed as separate layers to collectively form the covering or coating. The inner and outer layers may be formed from the same material or different materials, as desired.

The covering or coating may span or be disposed within openings or interstices 114 defined between adjacent stent filaments or struts 112 of the scaffold structure. It can be appreciated that as an inner layer and an outer layer extend outwardly and inwardly, respectively, they may touch and/or form an interface region within the spaces 114 (e.g., openings, cells, interstices) in the wall of the scaffold structure of the stent 10. For example, the inner and outer layers may extend into the openings 114 defined between adjacent stent struts 112 and form an interface region. Further, the inner and outer layers may additionally extend between adjacent filaments or struts 112, thereby filling any space 114 between adjacent filaments or struts 112, and thus prevent tissue ingrowth into the lumen of the stent 10.

In some embodiments, the covering or coating may extend along an entire length of the stent 10 from the first end 104 to the second end 106. In other embodiments, the covering or coating may extend along only a portion of the length of the stent 10. For example, the covering or coating may extend along only a portion of the length of the central or body portion 102, such as from a location spaced away from the first end 104 of the stent 10 toward the second end 106 of the stent 10 to a location spaced away from the second end 106 of the stent 10 toward the first end 104 of the stent 10 such that the first end 104 and the second end 106 of the stent 10 are free from the covering or coating, with only the central portion 102, or a portion thereof, having the covering or coating. Thus, a first end region proximate the first end 104 and/or a second end region proximate the second end 106 may be uncovered, while an intermediate region along the central portion 102, or a portion thereof, may be covered with a covering or coating. It is contemplated that the length of the uncoated end regions and the coated body region 102 may be determined based on the desired application, implant location, etc. In other instances, the covering or coating may extend from the first end 104 toward the second end 106, without extending all the way to the second end 106, leaving a second end region proximate the second end 106 uncovered. In other instances, the covering or coating may extend from the second end 106 toward the first end 104, without extending all the way to the first end 104, leaving a first end region proximate the first end 104 uncovered. In some examples, the stent 10 may be fully coated with openings for fluid flow processed into portions of the coating.

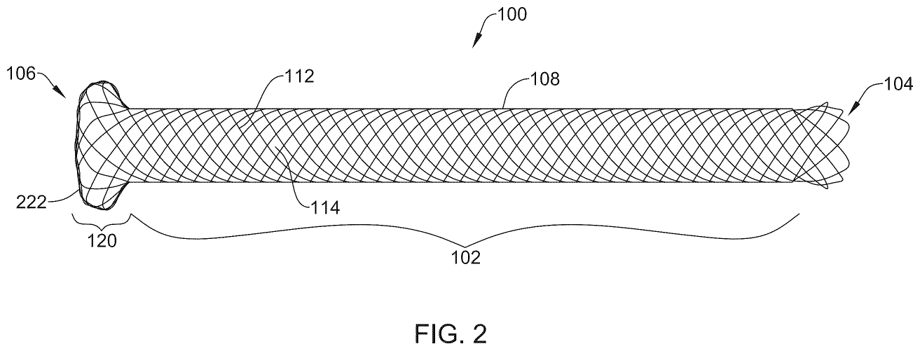

FIG. 2 illustrates an example stent 100 similar to the stent 10 in FIG. 1, in many respects. Accordingly, the discussion above regarding aspects of the stent 10 is equally applicable to the stent 100, and will not be repeated herein. Unlike the stent 10 of FIG. 1, the stent 100 of FIG. 2 includes a reinforced annular rim around the circumference of the shoulder or flared region 120 at the second end 106. The reinforced annular rim may be an area of the shoulder or flared region 120 in which the shoulder or flared region 120 has an area of condensed wire mass 222, for example. Though only shown at the second end 106 of the stent 100, it is contemplated that in some embodiments the first end 104 may also include a shoulder region 120 including a reinforced annular rim, such as an area of condensed mass 222. This condensed wire mass 222 may provide additional rigidity to the second end 104 resulting in additional forces in traditional pull-out-test methods, which are strongly linked to lower stent migration rates. Additional wire mass formed around the circumference of the shoulder or flared region 120 at the second end 106 may also increase the distance required for the stent to travel before reaching “peak force”, or the force required to cause stent migration. Said differently, this area of increased wire mass 222 may prevent the stent 100 from migrating after placement and causing damage to surrounding tissues.

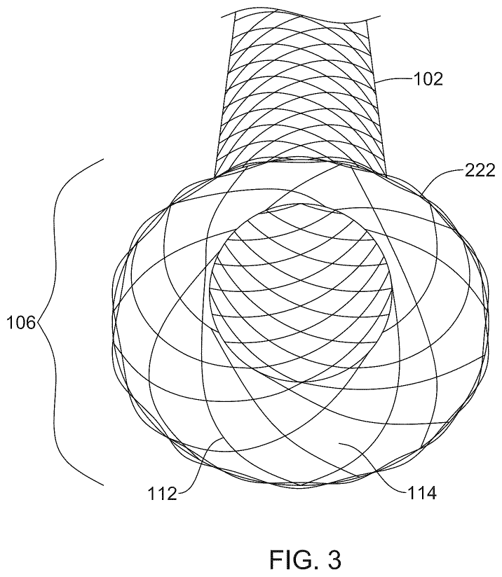

FIG. 3 illustrates a perspective view of the second end 106 of the stent of FIG. 2. The area of condensed wire mass 222 may form a ring around the outer circumference of the second end 106 at the opening or circumferential rim into the lumen of the stent 100. As shown, only the rim of the shoulder or flared region 120 (at the radial widest extent of the shoulder or flared region 120) forms the area of condensed wire mass 222, with the more medial portion of the shoulder region 120 (i.e., toward the central portion 102, and thus toward the first end 104) having the same scaffolding structure as the rest of the stent 100. In some embodiments, the length and/or diameter of the shoulder region 120 and second end 106 may be adjusted to increase or decrease the amount of wire concentrated within the area of condensed wire mass 222.

In some instances, the area of condensed wire mass 222 may be formed by compressing the existing stent struts or filaments 112 of the shoulder or flared region 120. The compressing of the filaments towards one another around the circumferential rim forms the ring of condensed wire mass 222 around the outer circumference of the second end 106.

Alternatively, or additionally to compressing the filaments 112 to a tighter density, in some instances additional wire filament(s) can be arranged around the rim of the shoulder or flared region 120 at the second end 106 (e.g., woven into the outer ring portion forming the annular rim of condensed wire mass 222 to add more wire mass. The added wire(s) may be the same wire as the stent scaffolding 112, or the added wire(s) may have a different diameter, material composition, etc. For instance, a wire may be woven through the cell openings or interstices 114 between adjacent filaments 112 around the circumference at the second end 106 of the shoulder or flared region 120 to form the annular ring of condensed wire mass 222. Though only one diameter is shown, it is contemplated that the diameter circumferential rim defining the ring of condensed wire mass 222 of the stent 100 and/or shoulder or flared region 120 may be increased or decreased to accommodate different anatomies and target locations within a body lumen.

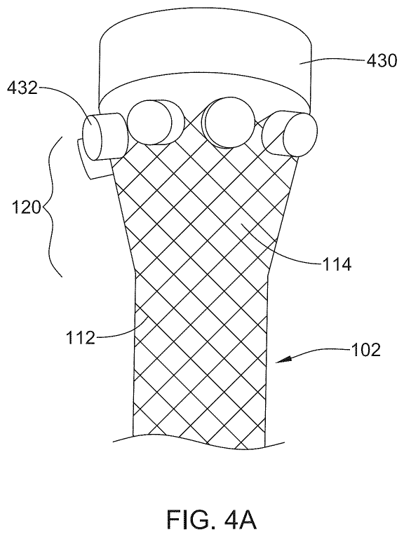

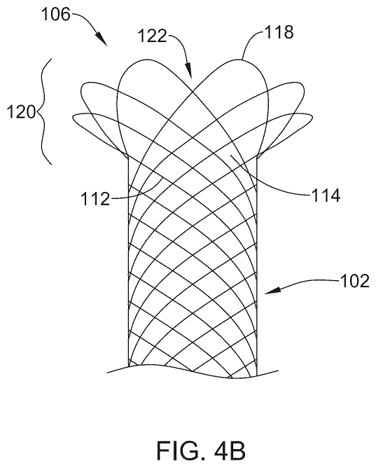

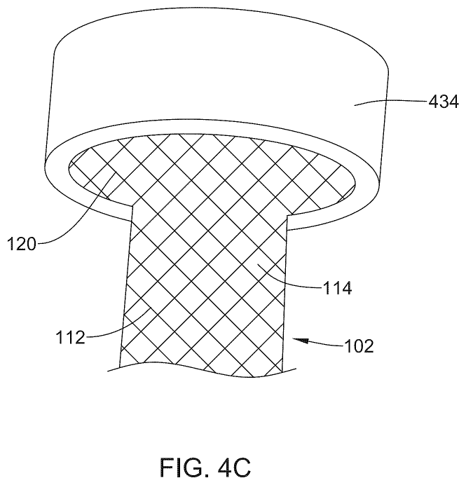

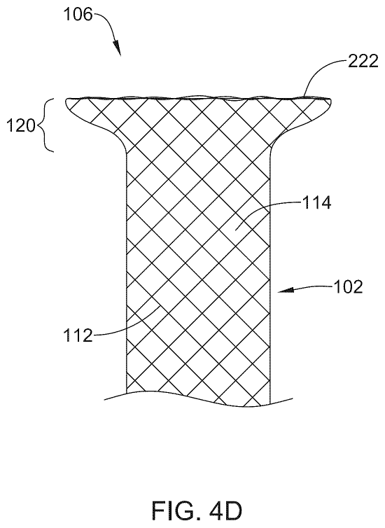

FIGS. 4A-4D illustrate one method of forming the ring of condensed wire mass 222. With reference to FIG. 4A, the stent 100 may be woven, braided, or knitted around a shaping mechanism or mandrel 430. The shaping mandrel 430 shown herein forms a flange shaped shoulder region 120, but it is contemplated that the shoulder region 120 may take any other shape e.g. oval, spheroid, hemispherical, tubular etc. Said differently, a cross section of the second end 106 and shoulder region 120 may take any shape desired. As discussed in more detail above, the stent 100 may be formed of one, or any number of filaments 112, woven around a shaping mandrel. The shaping mandrel 430 may have bumps, hooks, groove, notches, or the like for holding filaments 112 in place throughout the weaving process. For instance, the filaments 112 may be wrapped or bent around a plurality of posts 432 to form a plurality of loops at the second end 106 of the stent 100. The filaments 112 may be a superelastic and/or shape memory material, and/or may be heat set, such that the central portion 102 of the stent 100 and shoulder region 120 retain their shape after removal of the shaping mandrel 430.

As shown in FIG. 4B, after removal of the flange shaping mandrel 430, the shoulder or flared region 120 retains a general funnel or flared shape in which the shoulder or flared region 120 tapers radially outward toward the second end 106. The formed loops of the filaments (that were formed by bending the filaments 112 around the posts 432) define apexes 118 where the filaments 112 change direction and extend toward the first end 104 of the stent 100. The base of each of the loops, opposite the apex 118, may be a cross-over point 122 wherein two filaments 112 cross over one another. The loops, between the initial row of cross-over points 122 and the apexes 118, may define an end-most circumferential row of interstices 114. The apexes 118 of the plurality of wire loops are arranged around the circumferential rim. The apexes 118 of the loops of the filaments 112 at the end of the stent 100 are spaced apart, and are not concentrated. As shown, there are gaps between adjacent apexes 118 of the loops of the filaments 112 around the circumference of the stent 100 at the second end 106. Furthermore, there may be an initial longitudinal distance between the apexes 118 and the first cross over point 122 where the filaments 112 forming the loops 118 initially cross over one another to re-enter the braided pattern/configuration of the tubular member of the stent 100.

To form the area of condensed wire mass 222, a cap 434 is loaded onto the second end 106 of the stent, as shown in FIG. 4C. This cap 434 compresses at least a portion of the filaments 112 making up the shoulder or flared region 120. In some embodiments, the cap 434 may be configured to compress only a small portion of the shoulder or flared region 120. Though not shown, it is contemplated that the cap 434 may be shaped and sized to compress any number of filaments 112. Accordingly, the apexes 118 of the loops of the filaments 112 may be compressed toward the initial cross-over points 112, thus reducing the longitudinal distance between the apexes 118 and the cross-over points 122 of the end-most circumferential row of interstices 114. In some instances, the apexes 118 may be axially compressed toward the initial cross-over points 122 such that the apexes 118 are juxtaposed with the cross-over points 122, substantially reducing the cross-sectional area of the end-most circumferential row of interstices 114. Accordingly, the end-most circumferential row of interstices 114 may have a substantially smaller cross-sectional area than the interstices 114 along the central or body region 102 of the stent 100. It is noted that the circumferential rim has a first diameter in the as-braided configuration and a second diameter in the heat-set configuration, wherein the second diameter is greater than the first diameter.

The area of increased wire mass 222 may contain any amount of material depending on the length and diameter of the shoulder or flared region 120, as well as the shape and size of the cap 434. Once the cap 434 has been loaded onto the stent 100, the cap 434 and stent 100 are both heated to set the shape of the outer ring of condensed wire mass 222. The cap 434 may then be removed, and as shown in FIG. 4D, the stent 100, including the shoulder or flared region 120 and circumferential ring of condensed wire mass 222, all retain their shape upon removal of the shaping mandrel 430 and cap 434, referred to as a heat-set configuration.

It is contemplated that the scaffold structure, e.g., the filaments and/or struts, of the stent can be made from a number of different materials such as, but not limited to, metals, metal alloys, shape memory alloys, and/or polymers, as desired, enabling the stent to be expanded into shape when accurately positioned within the body. In some instances, the material may be selected to enable the stent to be removed with relative ease as well. For example, the stent can be formed from alloys such as, but not limited to, nitinol and Elgiloy®. Depending on the material selected for construction, the stent may be self-expanding or require an external force to radially expand the stent. In some embodiments, filaments may be used to make the stent, which may be composite filaments, for example, having an outer shell made of nitinol and having a platinum core. It is further contemplated that the filaments of the stent may be formed from polymers including, but not limited to, polyethylene terephthalate (PET), or a bioabsorbable polymeric material, if desired.

It will be understood that the dimensions described in association with the above figure are illustrative only, and that other dimensions of slits and filter sheaths are contemplated. The materials that can be used for the various components of the stent for capturing lesion particles (and/or other systems or components disclosed herein) and the various elements thereof disclosed herein may include those commonly associated with medical devices. For simplicity purposes, the following discussion refers to the stent (and variations, systems or components disclosed herein). However, this is not intended to limit the devices and methods described herein, as the discussion may be applied to other elements, members, components, or devices disclosed herein.

In some embodiments, the stent (and variations, systems or components thereof disclosed herein) may be made from a metal, metal alloy, ceramics, zirconia, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-clastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-clastic nitinol may also be termed “substantially” linear elastic and/or non-super-elastic nitinol.

In some cases, linear elastic and/or non-super-clastic nitinol may also be distinguishable from super elastic nitinol in that linear clastic and/or non-super-elastic nitinol may accept up to about 2-5% strain while remaining substantially elastic (e.g., before plastically deforming) whereas super clastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear clastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. For example, across a broad temperature range, the linear clastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the stent (and variations, systems or components thereof disclosed herein) may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the stent (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent (and variations, systems or components thereof disclosed herein) to achieve the same result.

In some embodiments, the stent (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some embodiments, the stent materials can be blended with a liquid crystal polymer (LCP). For example, the mixture can contain up to about 6 percent LCP.

In some embodiments, the stent (and variations, systems or components thereof disclosed herein) may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vascoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent, comprising:
an elongate tubular member having a first end, a second end, and a lumen extending from the first end to the second end, the tubular member comprising:
a scaffolding formed of a plurality of interwoven wires defining a plurality of interstices;
a first end region extending to the first end,
a second end region extending to the second end, the second end region forming a retention member; and
a body region extending between the first end region and the second end region;
wherein a diameter of the retention member is greater than a diameter of the body region; and
wherein a circumferential rim of the retention member includes an area of increased scaffolding density relative to a remainder of the first end region.

2. The stent of claim 1, wherein the area of increased scaffolding density forms an annular ring of the interwoven wires disposed around an outer circumference of the second end of the tubular member.

3. The stent of claim 2, wherein the annular ring of interwoven wires includes a plurality of wire loops extending around the circumferential rim.

4. The stent of claim 3, wherein each of the plurality of wire loops includes an apex and a base opposite the apex, wherein the base is a cross-over point of a pair of the plurality of interwoven wires.

5. The stent of claim 4, wherein the apex of each of the plurality of wire loops is juxtaposed with the base of the associated wire loop.

6. The stent of claim 4, wherein each of the plurality of wire loops has an as-braided configuration in which the apex is spaced apart from the base of the associate wire loop by a first distance, and wherein each of the plurality of wire loops has a heat-set configuration in which the apex is juxtaposed with the base of the associated wire loop a second distance, wherein the first distance is greater than the second distance.

7. The stent of claim 6, wherein the apexes of the plurality of wire loops are arranged around the circumferential rim.

8. The stent of claim 7, wherein the circumferential rim has a first diameter in the as-braided configuration and a second diameter in the heat-set configuration, wherein the second diameter is greater than the first diameter.

9. The stent of claim 3, wherein the plurality of wire loops defines an end-most circumferential row of interstices, wherein the interstices in the end-most circumferential row of interstices have a smaller cross-sectional area than interstices along the body region.

10. The stent claim 2, wherein the area of increased scaffolding density includes an additional wire circumferentially woven into the scaffolding around the circumferential rim.

11. The stent of claim 1, wherein the retention member is substantially flange shaped.

12. The stent of claim 1, wherein the first end region of the tubular member forms a retention member, wherein a diameter of the retention member of the first end region is greater than the diameter of the body region.

13. The stent of claim 12, wherein a circumferential rim of the retention member of the first end region includes an area of increased scaffolding density.

14. A stent, comprising:
an elongate tubular member having a first end, a second end, and a lumen extending from the first end to the second end, the tubular member comprising:
a scaffolding formed of a plurality of interwoven wires defining a plurality of interstices;
a first end region extending to the first end,
a second end region extending to the second end, the second end region being a flared region tapered radially outward to an outermost circumferential rim at the second end; and
a body region extending between the first end region and the second end region;
wherein a diameter of the circumferential rim is greater than a diameter of the body region;
wherein the plurality of interwoven wires forms a plurality of wire loops extending around the circumferential rim;
wherein each of the plurality of wire loops includes an apex and a base opposite the apex, wherein the base is a cross-over point of a pair of the plurality of interwoven wires; and
wherein the apex of each of the plurality of wire loops is juxtaposed with the base of the associated wire loop.

15. The stent of claim 14, wherein the plurality of wire loops defines an end-most circumferential row of interstices, wherein the interstices in the end-most circumferential row of interstices have a smaller cross-sectional area than interstices along the body region.

16. The stent of claim 14, wherein each of the plurality of wire loops has an as-braided configuration in which the apex is spaced apart from the base of the associate wire loop by a first distance, and wherein each of the plurality of wire loops has a heat-set configuration in which the apex is juxtaposed with the base of the associated wire loop a second distance, wherein the first distance is greater than the second distance.

17. The stent of claim 16, wherein the apexes of the plurality of wire loops are arranged around the circumferential rim.

18. The stent of claim 17, wherein the circumferential rim has a first diameter in the as-braided configuration and a second diameter in the heat-set configuration, wherein the second diameter is greater than the first diameter.

19. A method of forming a stent, the method comprising:
braiding a plurality of wires to form a tubular scaffolding comprising a plurality of loops arranged around a circumferential rim of the tubular scaffolding;
placing a cap over the circumferential rim of the tubular scaffolding;
compressing the plurality of loops arranged around the circumferential rim of the tubular scaffolding to form a condensed wire mass around the circumferential rim of the tubular scaffolding;
heat setting the condensed wire mass; and
removing the cap.

20. The method of claim 19, wherein each of the plurality of wire loops includes an apex and a base opposite the apex, wherein the base is a cross-over point of a pair of the plurality of interwoven wires; and
wherein the apex of each of the plurality of wire loops is juxtaposed with the base of the associated wire loop after the heat setting step.
